(19) United States
(12) Patent Application Publication
(10) Pub. No.: US 2006/0058826 A1
(43) Pub. Date: Mar. 16, 2006

(54) Tissue cavitation device

(76) Inventors: Avery J. Evans, Charlottesville, VA (US); Josephine A. Evans, Charlottesville, VA (US)

Correspondence Address: COLLIER SHANNON SCOTT, PLLC, 3050 K STREET, NW, SUITE 400, WASHINGTON, DC, 20007, US

(21) Appl. No.: 11/210,867

(22) Filed: Aug. 25, 2005

Related U.S. Application Data
(60) Provisional application No. 60/604,021, filed on Aug. 25, 2004.

(51) Int. Cl. A61B 17/32

(52) U.S. Cl. 606/170

(57) ABSTRACT

An embodiment of the present invention is a tissue cavitation device, comprising a cannula, an insertion tube, and a cutting shaft, wherein a portion of the cutting shaft is slidably engaged within the cannula and a portion of the cannula is slidably engaged within the insertion tube. The cutting shaft may have a support material in communication with the cutting shaft to modify the physical characteristics of the cutting shaft.

### CROSS REFERENCE TO RELATED APPLICATIONS

The present invention relates to, and is entitled to the benefit of the earlier filing date and priority of, Application No. 60/604,021, filed on Aug. 25, 2004, which is herein incorporated by reference as if fully set forth.

### FIELD OF THE INVENTION

The present invention relates generally to an apparatus and method to create a cavity within a tissue.

### BACKGROUND OF THE INVENTION

Minimizing trauma to patients during medical procedures is a guiding principle of physicians. For surgeons, this principle encompasses procedures for minimizing the surgical trauma to the patient while at the same time achieving the goals of the surgical procedure. Some practical applications of this principle have achieved new levels of success with the technological advancements of minimally invasive surgical procedures. For example, techniques employing direct and indirect visualization methods allow surgeons to perform surgical procedures through smaller incisions in the body. These techniques have been refined to successful clinical applications. Orthopedic, gynecologic, and thoracoabdominal surgical techniques have been revolutionized by progress in these surgical techniques.

In furtherance of this principle, the formation of a cavity within either soft or hard tissue, including bone, is often necessary during surgery. These cavities may be formed by creating an opening in the target tissue, wherein a device may be inserted into the tissue to form a cavity that is larger than the opening required to access the tissue. For example, certain surgical procedures may require the surgeon to create a cavity in tissue such as bone.

Bones provide structural and protective support. Two types of bone tissue enable bones to be both rigid enough to withstand immense forces and light enough to respond to attached muscles. Cortical bone provides rigidity while trabecular or cancellous bone provides strength and elasticity. Diseases like osteoporosis and metastatic breast cancer, as well as other tumors like hemangiomas, weaken trabecular bone and lead to fractures.

The most common fracture is a vertebral compression fracture. There are more than 700,000 vertebral compression fractures in the USA annually, resulting in over 200,000 hospitalizations. These fractures are painful in more than 80% of patients. Spinal deformity and pain impair function and decrease mobility. Decreased activity leads to more bone loss. Kyphosis may occur, compressing the abdomen and the chest and leading to decreased appetite, sleep disorders, and decreased lung capacity.

Traditional treatments for vertebral compression fractures have offered only pain management and rest, exacerbating bone loss. Vertebroplasty, a procedure involving the percutaneous injection of polymethylmethacrylate (PMMA) into the fractured vertebral body has been practiced in the Untied States for over 10 years. Vertebroplasty has been shown to have a statistically significant improvement on patients' pain and mobility levels after vertebral compression fracture. Vertebroplasty is cost-effective, usually performed on an outpatient basis without general anesthesia.

A more recent procedure, kyphoplasty, involves introducing a balloon device into the fractured vertebral body. Inflating the balloon compresses the trabecular bone and creates a cavity with the vertebral body. The balloon is removed, and the cavity is then filled with bone cement.

Kyphoplasty claims to restore height to the vertebral body and, like vertebroplasty, prevents further fracture or movement of the defective trabecular bone. The larger incision and instruments required for kyphoplasty increase cost and may increase morbidity. There is no level one study corroborating kyphoplasty's effectiveness in restoring vertebral height. Neither kyphoplasty or vertebroplasty removes the diseased, or otherwise undesired, bone or bone fragments from the vertebral body. Examples of procedures requiring such a cavity in bone include, but are not limited to, hip augmentation, vertebroplasty, and removal of tumor or other pathologic process from any bony structure. What is needed in the industry is an apparatus and method to create a cavity in a tissue, such as, but not limited, to bone, that is minimally invasive to the patient, and allows removal of the undesired tissue.

The discussion above provides an example of the need for a minimally invasive apparatus and method to create a cavity in bone tissue, but embodiments of the present invention are not limited to use in bone, and may be used in any other suitable tissue type. Additional advantages of various embodiments of the invention are set forth, in part, in the description that follows and, in part, will be apparent to those of ordinary skill in the art from the description and/or from the practice of the invention.

### SUMMARY

Embodiments of the present invention relate to an apparatus and method for forming a cavity within tissue, wherein the apparatus may be inserted into the tissue to form a cavity within the tissue that is larger than the opening required to insert the apparatus and perform the procedure. An embodiment of the present invention is a tissue cavitation device, comprising a cannula, an insertion tube, and a cutting shaft, wherein a portion of the cutting shaft is slidably engaged within the cannula and a portion of the cannula is slidably engaged within the insertion tube. The cutting shaft may have a support material in communication with the cutting shaft to modify the physical characteristics of the cutting shaft. The device may further comprise a drive unit, a sleeve, and/or a fluid applicator. The sleeve and/or the cannula may have a first position encompassing at least the portion of the cutting shaft and a second position wherein at least a portion of the cutting shaft that was encompassed in the first position is not encompassed in the second position.

Tissue is defined herein for the purposes of this application to mean a collection of similar cells and the intercellular substances surrounding them, including the epithelium, connective tissues (including bone, blood, and cartilage), and nerve tissue.

Additional advantages of the invention will be set forth in part in the description that follows, and in part will be obvious from the description, or may be learned by practice of the invention. The advantages of the invention will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments of the invention and together with the description, serve to explain the principles of the invention. Where appropriate, the same reference numerals refer to the same or similar elements.

FIG. 1 is an elevational view of an embodiment of the device for producing a cavity according to an embodiment of the present invention.

FIG. 2 is an axial view of an embodiment of the device for producing a cavity according to an embodiment of the present invention.

FIG. 3 is a cross sectional view of an embodiment of the device for producing a cavity according to an embodiment of the present invention.

FIG. 4 is an elevational view of an embodiment of the device in two different states according to an embodiment of the present invention.

FIG. 5 is an elevational view of an embodiment of the device according to an embodiment of the present invention.

FIG. 6 is a drill bit for an embodiment of the device according to an embodiment of the present invention.

FIG. 7 is a second shaft for an embodiment of the device according to an embodiment of the present invention.

### DETAILED DESCRIPTION

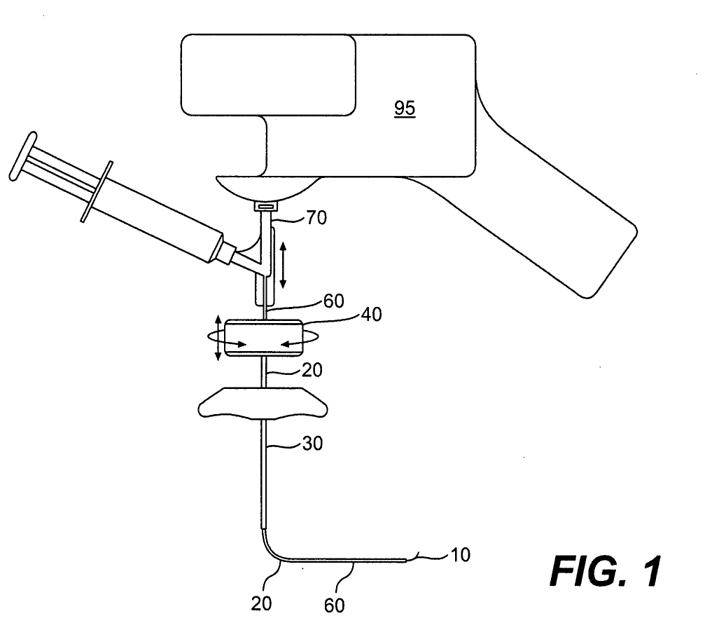

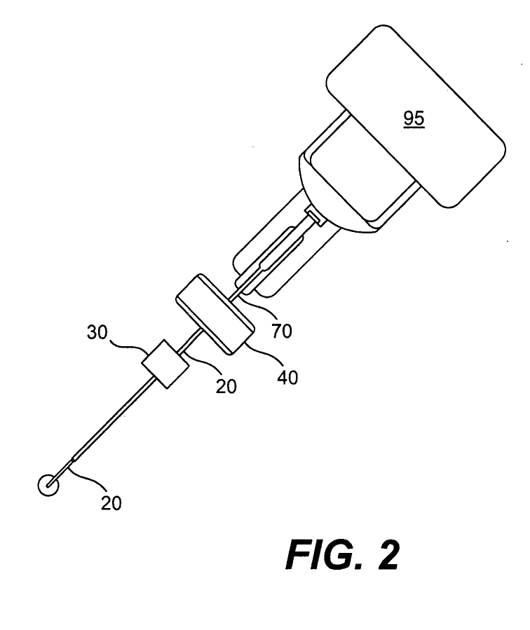

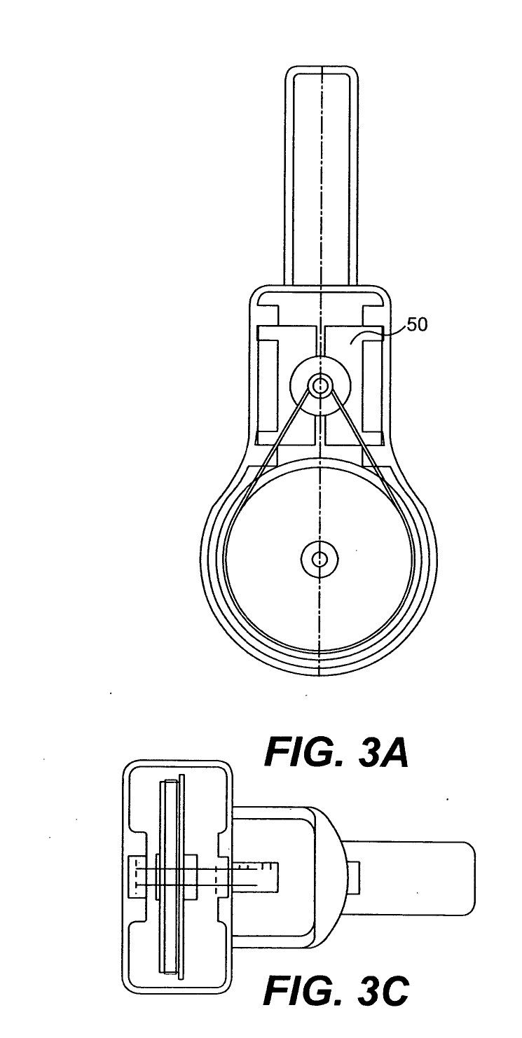

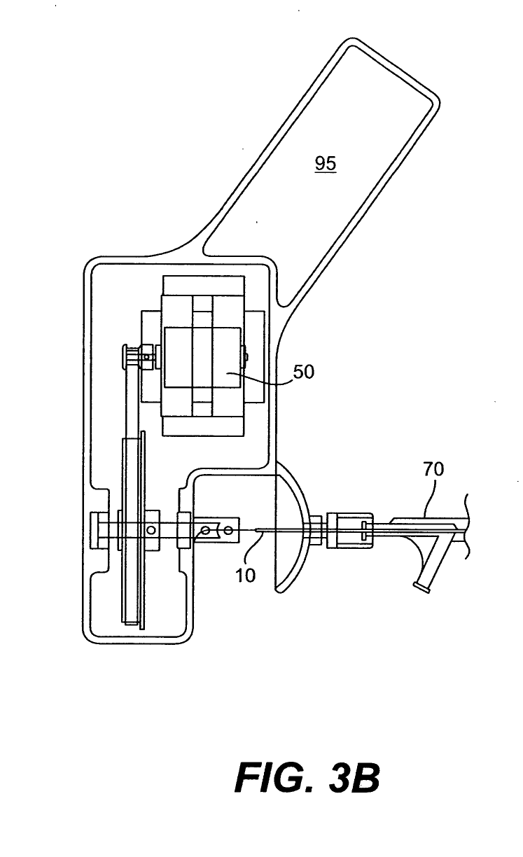

As shown in FIGS. 1 and 2, an embodiment of the cavitation system of the present invention comprises cutting shaft 10, cannula 20, insertion tube 30, and drive unit 50, wherein the proximal end of cutting shaft 10 is releasably attached to drive unit 50 and the distal portion of the cutting shaft is slidably engaged in cannula 20, and the distal portion of cannula 20 is slidably engaged in insertion tube 30. Proximal and distal are used to describe either ends or portions of elements, wherein proximal indicates the end or portion nearest the operator, and distal indicates the end or portion away from the operator.

Cutting shaft 10 may be comprised of nitinol wire or any other suitable material. Examples of such materials include, but are not to, polytetrafluoroethylene (PTFE), polyetheretherketone (PEEK), Teflon@, Kevlar®, steel, stainless steel, or any other suitable material. Cutting shaft 10 may be in a range of about 0.001 inches to 0.3 inches in diameter and may be solid or be tube-like with an open bore.

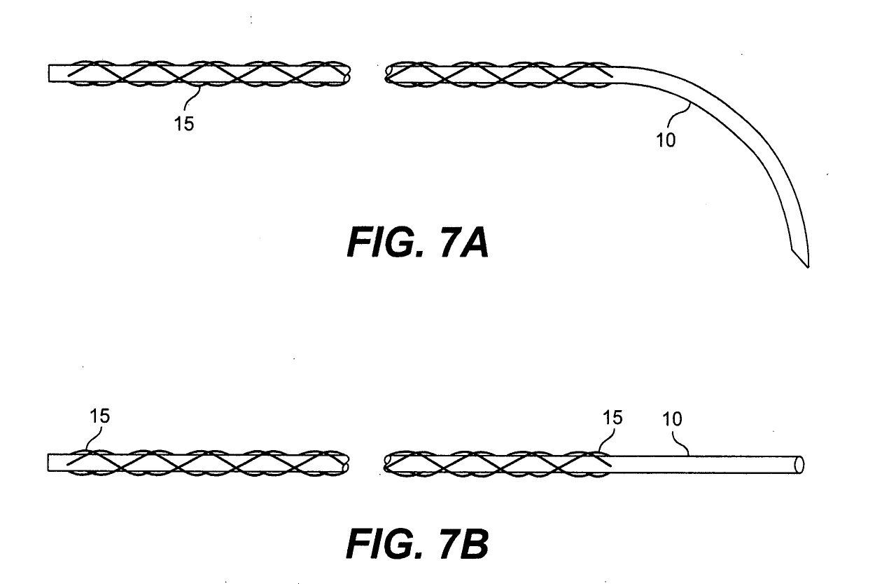

As shown in FIG. 7, an embodiment of cutting shaft 10 may have a support material 15 overlaid on all or on a distal portion of cutting shaft 10. Support material 15 may aid in tissue cavitation, and may be used to modify the rigidity or strength characteristics of cutting shaft 10 to allow cutting shaft 10 to remove certain types or densities of tissue and reduce or minimize damage to other tissue, such as, but not limited to, healthy tissue. For the purposes of the following discussions, the definition of cutting shaft 10 is meant to include both cutting shaft 10 alone and cutting shaft 10 in combination with support material 15.

For example, one embodiment of the present invention allows the operator to create a cavity in cancellous bone but leave the cortical bone intact, based on the difference in tissue density. The vertebral body is essentially a “box” filled with bone marrow. Depending on the characteristics of cutting shaft 10 with or without support material 15 the operator can preferentially macerate and remove the bone marrow, and leave the cortical bone of the vertebral body intact. Other important structures of the spine: the cord, the nerve roots, the blood vessels, including, but not limited to, the aorta, are located outside the vertebral body. The characteristics of cutting shaft 10 with or without support material 15 provide the ability to hollow out the vertebral body and leave other structures substantially intact.

Support material 15 may be comprised of stainless steel, such as, but not limited to a stainless steel braid, such as a T304 stainless steel braid, or any other suitable material, such as, but not limited to, Kevlar®, Teflon®, nylon, and any other suitable plastics, such as, but not limited to, PTFE and PEEK. Support material 15 may be braided around, molded in, weaved through, or lining cutting shaft 10 or in any other pattern. In an embodiment, support material 15 is braided; however, any type of braiding or covering, solid or with an open pattern, could be used. Further, support material 15 may be overlaid in any pattern on all or on the distal portion of cutting shaft 10, including where support material 15 completely covers or overlays at least a portion of cutting shaft 10. It is envisioned that support material 15 may also cover the entire cutting shaft 10 or that more than one support material 15 can be used. Cutting shaft 10 including support material 15 is dimensioned to pass slidably through the interior of cannula 20 and insertion tube 30.

By way of example, a 0.021 inch diameter cutting shaft 10 may be braided with support material 15 comprising 1×4×0.008 inch T304 stainless steel braid where the braid assumes the pattern shown in FIG. 7. While support material 15 may be soldered to cutting shaft 10, any suitable means of attachment such as, but not limited to, welding, heat shrink tubing, screw in fitting, and metal sleeves could be used to attach support material 15 to cutting shaft 10. In an embodiment, cutting shaft 10 is round and has a diameter in a range of about 0.001 inches to about 0.3 inches. In this embodiment, cutting shaft 10 has a circular cross section, but all other cross sections, such as square, are within the scope of the invention.

Support material 15 may also be attached to only the distal portion of cutting shaft 10. For example, a portion of support material 15 comprising a steel braid could be attached to the distal end of cutting shaft 10 that is inserted into cannula 20 and insertion tube 30. Further, support material 15 would not have to be directly connected to cutting shaft 10 and could be connected by an intermediary material (not shown). Further, support material 15 could be attached at other surfaces on cutting shaft 10, including, but not limited to, any surface along the side of cutting shaft 10 in one ore more locations, or in one embodiment, inside the bore of cutting shaft 10. However, cutting shaft 10 and the attached support material 15 will still pass through the interior of cannula 20.

In an embodiment, cutting shaft 10 with or without support material 15 may also be comprised of a cutting tip at its distal end (not shown). The cutting tip may be of any suitable material, such as, but not limited to, metal or plastic, and may be of any suitable shape, including, but not limited to, a sphere, disc, arrow, spikes, triangle, barbs, or any other suitable shape. The cutting tip may aid in cutting, macerating, or removing specific types of tissue and may add to the centrifugal force generated by the spinning of cutting shaft 10.

Support material 15 may be aligned with cutting shaft 10 so that the distal ends of support material 15 and cutting shaft 10 are the same length, or the distal end of support material 15 may extend beyond the distal end of cutting shaft 10 or vice versa. In an embodiment, the distal end of support material 15 may be slidably engaged with cutting shaft 10, and adjustable by the operator as needed.

In an embodiment any or all of cutting shaft 10 and support material 15 may be coated with PTFE, either individually or as a unit.

A portion of cannula 20 is slidably engaged within insertion tube 30. Cannula 20 may be comprised of nitinol or any other suitable material. Cannula 20 may assume a predetermined curved position with in the patient. The proximal end of cannula 20 may be attached to hub 40 at a point proximal to where cannula 20 exits the proximal end of insertion tube 30. Hub 40 is affixed to cannula 20 and is used by the operator to slide and/or rotate cannula 20 into a desired position within the patient. As shown in FIGS. 1 and 2, by manipulating hub 40 a curved cannula 20 can be directed in any direction in, for example, a vertebral body, allowing cutting shaft 10 to be directed and allowing cutting in any direction and up and down multiple spinal levels.

Insertion tube 30 can be a trochar, cannula, needle, or any other suitable structure. In an embodiment of the present invention, insertion tube 30 is a needle in the range of about 20 gauge to about 6 gauge. In an embodiment of the present invention an 11 gauge spinal needle comprises a portion of insertion tube 30. The distal end of insertion tube 30 may be fluoroscopically guided into the desired location in the patient. Once in position, the distal end of cannula 20 may be introduced into the proximal end of insertion tube 30 and out through the distal end of insertion tube 30 and into the patient.

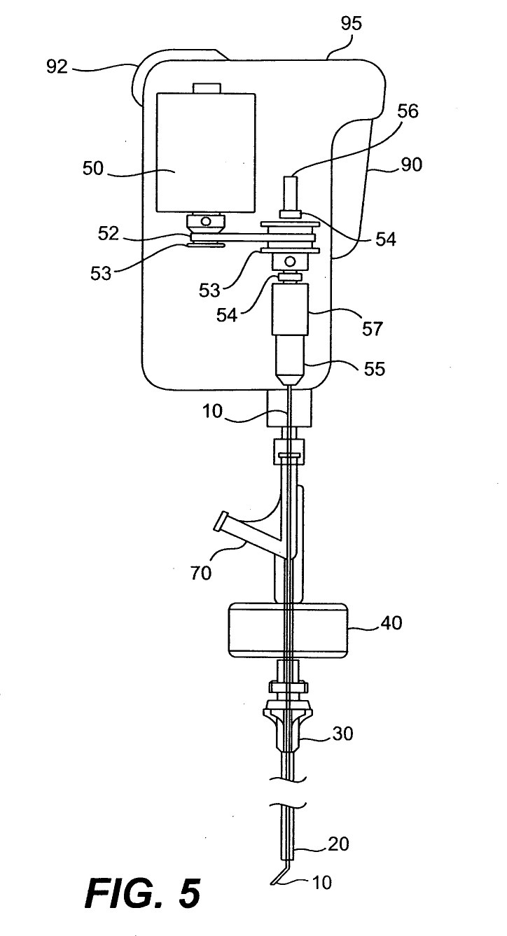

An embodiment of the present invention further comprises drive unit 50 which is releasably connected either directly or indirectly to the proximal end of cutting shaft 10 wherein drive unit 50 can be used to rotate cutting shaft 10. Drive unit 50 may be a small commercially available hand-held drill and can be battery operated; however numerous other options for either power (AC) or manual operation are known and would be suitable. Drive unit 50 may be variable speed and controlled by switch 92. As shown in FIG. 5, an embodiment of drive unit 50 may contain a DC motor which moves timing belt 52 through a series of pulleys 53 and further comprising a shaft 56 and bearings 54 mounted in housing 95 such that coupling 57 and collet 55 transfers rotation to cutting shaft 10. These mechanisms are known in the art. In an embodiment cutting shaft 10 is coupled to collet 55 which holds cutting shaft 10 in place. In other embodiments, cutting shaft 10 could be connected to a rotatable shaft by other means and cutting shaft 10 could be rotated directly or indirectly by the DC motor. Cutting shaft 10 also could be rotated by any other suitable means.

The distal portion of cutting shaft 10 with or without support material 15 may have a shape memory characteristic comprising a constrained first configuration substantially parallel to the distal end of cannula 20 or sleeve 60, and assume a relaxed second configuration that deviates from parallel when the distal end of cannula 20 or sleeve 60 is retracted and the distal end of cutting shaft 10 is exposed. In another embodiment, the distal portion of cutting shaft 10 does not posses a shape memory characteristic and upon retraction of cannula 20 or sleeve 60 retains its substantially parallel first configuration. Upon activation of drive unit 50, the rotation of cutting shaft 10 creates sufficient centrifugal force such that the distal exposed portion of cutting shaft 10 assumes the angular second configuration during rotation. The dimensions of the cavity may be controlled by the composition, speed of rotation and/or exposed length of cutting shaft 10, and the manipulation of housing 95 and hub 40.

An embodiment of the present invention may further comprise a sleeve 60, as shown in FIG. 1. Sleeve 60 may be comprised of a polymer, such as, but not limited to, PEEK and/or polyimide, or any other suitable material. The distal portion of cutting shaft 10 is slidably engaged within sleeve 60, and the distal portion of sleeve 60 is slidably engaged within cannula 20 and hub 40. As shown in FIG. 1, by manipulating hub 40 a curved cannula 20 can be directed in any direction in, for example, a vertebral body, allowing sleeve 60 for cutting shaft 10 and therefore cutting shaft 10 to be directed and allowing cutting in any direction and up and down multiple spinal levels.

In an embodiment shown in FIG. 1, the proximal end of sleeve 60 may be fluidity engaged with fluid applicator 70 to permit the application of a fluid inside sleeve 60. The fluid may be for lubrication and/or cooling and may be composed of, but is not limited to, water, saline, or any other suitable fluid. Fluid applicator 70 comprises a fitting, such as, but not limited to, a luer-lock fitting such that a syringe, tubing, or another type of applicator may be affixed to provide the fluid, and also comprises a sealing assembly such as, but not limited to, an “O” ring to prevent the fluid from entering housing 95 or drive unit 50. It is envisioned that other fluids may be injected through fluid applicator 70 into sleeve 60, including, but not limited to, cleansing, antibiotic, or other medicated solutions, and in the case of vertebroplasty, bone cement. Sleeve 60 may be used independently without fluid applicator 70 to provide an additional protective sleeve for cutting shaft 10 or for tubular shaft 80 and drill bit 85 discussed in the following paragraphs.

According to the method of an embodiment of the present invention, insertion tube 30 is inserted into the patient at the desired location. For example, in a surgical procedure involving the vertebra, the distal end of insertion tube 30 is placed through the pedicle into a vertebral body using x-ray, or any other suitable guidance mechanism. The distal end of cannula 20 is inserted into the proximal end of insertion tube 30. In one embodiment, the distal end of sleeve 60 may be inserted into cannula 20 and into the patient. Tubular shaft 80 with drill bit 85 attached to its distal end is inserted into the proximal end of sleeve 60, with drill bit 85 and the distal portion of tubular shaft 80 entering the patient. In an embodiment of the method, sleeve 60 containing tubular shaft 80 with drill bit 85 may be inserted into cannula 20 and insertion tube 30. Tubular shaft 80 and drill bit 85 may be composed of stainless steel or any other suitable material. The proximal end of tubular shaft 80 remains outside of the patient and is releasably attached either directly of indirectly to drive unit 50, or by any other suitable type of drive unit, such as a small hand-held drill, or by any other suitable method. Drive unit 50 is actuated and tubular shaft 80 containing drill bit 85 spins at a user-controlled rate and drill bit 85, sleeve 60, and cannula 20 are advanced allowing drill bit 85 to create the aperture needed to insert cutting shaft 10. Fluoroscopic guidance may be used, or any other suitable guidance, if necessary, to guide drill bit 85 as an aperture is created in the desired tissue.

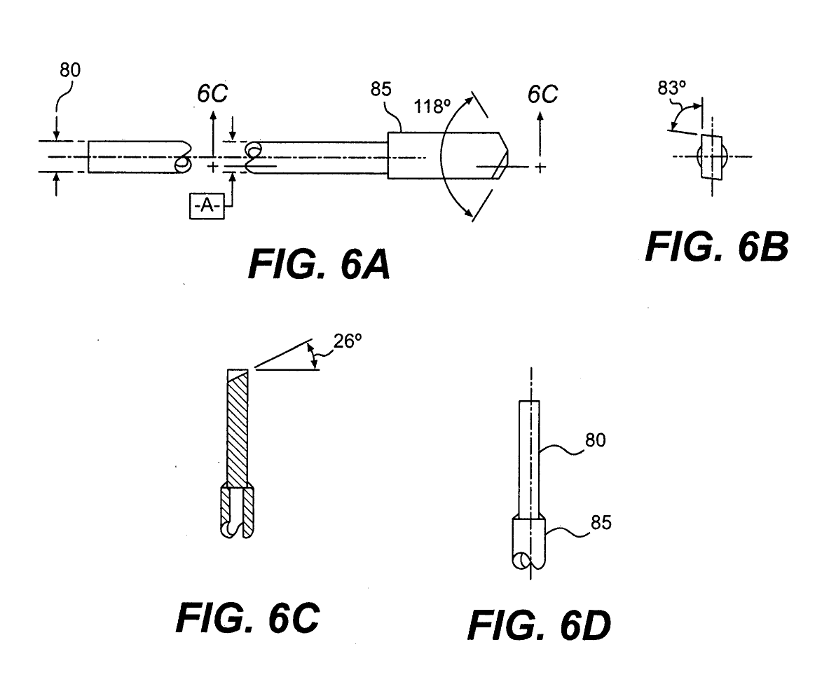

An example of a drill bit that could be used in this embodiment of the invention is shown in FIG. 6. For example, for use in bone tissue, drill bit 85 may be similar to a masonry drill bit. However, any drill bit suitably adapted for the purpose may also be used.

Cannula 20 and sleeve 60 are directed and advanced as drill bit 85 advances through the tissue. For example, drill bit 85 may create an aperture in the range of about 0.8 mm to about 2.4 mm in diameter, and depending on the application, in a range from about 1.2 mm to 2.0 mm in diameter.

Tubular shaft 80 and drill bit 85, or tubular shaft 80, drill bit 85 and sleeve 60, are removed from cannula 20 and the distal end of cutting shaft 10 is inserted into cannula 20 and/or sleeve 60 and into the patient. The proximal end of cutting shaft 10 is releasably attached either directly or indirectly to drive unit 50. This can be the same drive unit after detaching tubular shaft 80 or may be a different drive unit. Drive unit 50 is actuated, spinning cutting shaft 10. Drive unit 50 may be variable speed permitting the operator to adjust the speed of rotation of cutting shaft 10 as desired. Rotating cutting shaft 10 is advanced through cannula 20 and/or sleeve 60 and the distal portion of cutting shaft 10 is exposed into the tissue to be removed. The distal portion of cutting shaft 10 may assume an angled second configuration due to memory shape characteristics of cutting shaft 10 and/or support material 15, or in the absence of any memory shape characteristic, the assumption of the angled second configuration is based on the centrifugal force created by the rotation of the cutting shaft 10 and or support material 15. Cannula 20 may be manipulated by hub 40, thus exposing and directing cutting shaft 10, as discussed previously.

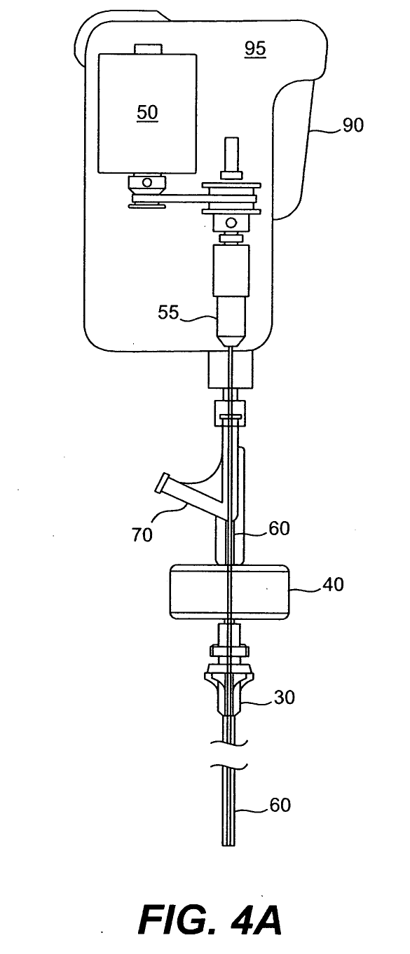

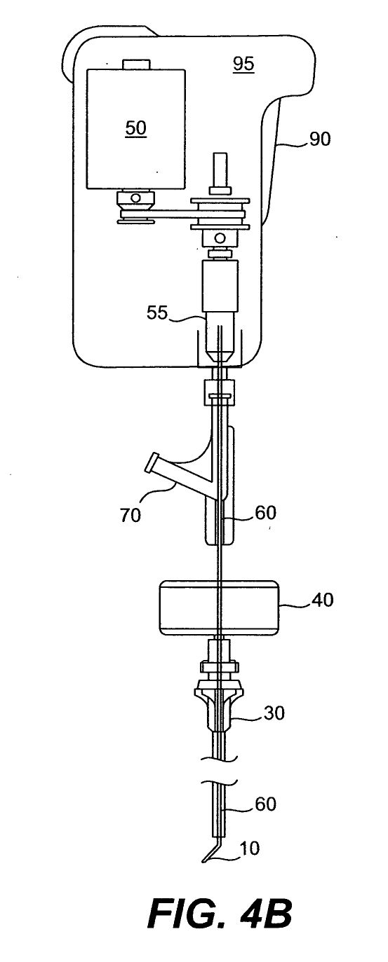

In an embodiment, housing 95 incorporates a mechanism to withdraw cannula 20 and/or sleeve 60 as a unit to expose cutting shaft 10 as shown in FIG. 4. A gear mechanism actuated by trigger 90 pulls cannula 20 and/or sleeve 60 into housing 95. Also, the entire housing 95 can be pushed forward or pulled backward, advancing cutting shaft 10 farther into the tissue, or pulling it back to withdraw from the tissue. For example, when working in the vertebra, the longer the length of cutting shaft 10 that is in the bone, and exposed beyond the distal end of the cannula 20 and/or sleeve 60, the greater the radius of its cutting path. As is seen in FIG. 1, a curved cannula allows sleeve 60 for cutting shaft 10, or in the absence of sleeve 60, allows cutting shaft 10 to be directed in any direction in the vertebral body by turning hub 40 for cannula 20. Also, an embodiment of the invention allows the operator to access vertebral bodies up and down the spine for several levels since sleeve 60 for cutting shaft 10 can be pushed through the disc spaces and into adjacent vertebral bodies.

The cutting properties of cutting shaft 10 may be adjusted by the composition of cutting shaft 10. Different materials may be used to create a cavity in different types of tissue, as discussed previously. In one example of using an embodiment of the present invention to create a cavity in bone tissue, the cutting element may be designed such that cutting shaft 10 when actuated by drive unit 50 will preferentially remove defective trabecular bone but will not substantially destroy normal cortical bone. For this application a nitinol cutting shaft 10 covered with a stainless steel braid support material 15 as discussed previously is suitable.

For example, one embodiment of the present invention allows the operator to create a cavity in cancellous bone but leave the cortical bone intact, based on the difference in tissue density. The vertebral body is essentially a “box” filled with bone marrow. Depending on the characteristics of cutting shaft 10 with or without support material 15 the operator can preferentially macerate and remove the bone marrow, and leave the cortical bone of the vertebral body intact. Other important structures of the spine: the cord, the nerve roots, the blood vessels, including, but not limited to, the aorta, are located outside the vertebral body. The characteristics of cutting shaft 10 with or without support material 15 provide the ability to hollow out the vertebral body and leave other structures substantially intact.

A cavity is created by a series of passes of the spinning cutting shaft 10. The desired diameter and shape of the cavity can be controlled by manipulating housing 95, drive unit 50 and hub 40. Once the cavity reaches the desired configuration, cutting shaft 10 may be removed. For example, within the vertebra, the diameter of the cavity may range from about 2.0 mm to 15 mm or greater as needed. A diameter of about 10 mm would be normal. Suction may be inserted through cannula 20, and/or sleeve 60, or through a second cannula (not shown) for the removal of tissue debris.

Numerous characteristics and advantages have been set forth in the foregoing description, together with details of structure and function. The novel features are pointed out in the appended claims. The disclosure, however, is illustrative only, and changes, may be made in detail, especially in matters of shape, size, and arrangement of parts, within the principle of the invention, to the full extent indicated by the broad general meaning of the terms in which the appended claims are expressed.

What is claimed is:

1. A tissue cavitation device, comprising:
a cannula,
an insertion tube,
and a cutting shaft,
wherein a portion of the cutting shaft is slidably engaged within the cannula and a portion of the cannula is slidably engaged within the insertion tube.

2. The tissue cavitation device of claim 1, wherein the cutting shaft further comprises a support material in communication with at least a portion of the cutting shaft.

3. The tissue cavitation device of claim 2, wherein the cutting shaft is comprised of nitinol.

4. The tissue cavitation device of claim 2, wherein at least a portion of the support material is soldered to the cutting shaft.

5. The tissue cavitation device of claim 2, wherein the support material is in communication with the portion of the cutting shaft that is inserted into a patient.

6. The tissue cavition device of claim 2, wherein the support material is in communication with the cutting shaft in a braided pattern.

7. The tissue cavitation device of claim 2, further comprising a fluid applicator to deliver a fluid to the tissue cavity.

8. The tissue cavitation device of claim 2, further comprising a sleeve having a first position encompassing at least the portion of the cutting shaft and a second position wherein at least a portion of the cutting shaft that was encompassed in the first position is not encompassed in the second position.

9. A tissue cavitation device, comprising:
a drive unit,
a cannula,
an insertion tube,
a cutting shaft,
wherein a distal portion of the cutting shaft is slidably engaged within the cannula and a portion of the cannula is slidably engaged within the insertion tube,
wherein the cutting shaft has a support material in communication with at least a portion of the cutting shaft, and
wherein the proximal end of the cutting shaft is releasably attached to the drive unit.

10. The tissue cavitation device of claim 9, wherein the cutting shaft is comprised of nitinol.

11. The tissue cavitation device of claim 9, wherein at least a portion of the support material is soldered to the cutting shaft.

12. The tissue cavitation device of claim 9, wherein the support material is in communication with the distal portion of the cutting shaft.

13. The tissue cavition device of claim 9, wherein the support material is in communication with the cutting shaft in a braided pattern.

14. The tissue cavitation device of claim 9, further comprising a fluid applicator to deliver a fluid to the distal end of the cutting shaft.

15. The tissue cavitation device of claim 9, further comprising a sleeve having a first position encompassing at least the portion of the cutting shaft and a second position wherein at least a portion of the cutting shaft that was encompassed in the first position is not encompassed in the second position.

16. A tissue cavitation device, comprising:
a drive unit,
an insertion tube,
a cannula slidably engaged within the insertion tube,
a sleeve slidably engaged within the cannula,
a cutting shaft slidably engaged within the sleeve,
wherein the cutting shaft further comprises a support material in communication with at least a portion of the cutting shaft, and
wherein the sleeve has a first position encompassing at least a portion of the cutting shaft and a second position wherein at least a portion of the cutting shaft that was encompassed in the first position is not encompassed in the second position.

17. The tissue cavitation device of claim 16, wherein the cutting shaft comprises nitinol.

18. The tissue cavitation device of claim 16, wherein at least a portion of the support material is soldered to the cutting shaft.

19. The tissue cavitation device of claim 16, wherein the support material is in communication with the distal portion of the cutting shaft.

20. The tissue cavitation device of claim 16, further comprising a fluid applicator to deliver a fluid to the distal end of the cutting shaft.

21. A method for creating a cavity in a tissue comprising:
inserting an insertion tube into a patient,
inserting a cannula into the insertion tube and into the patient,
inserting a cutting shaft having a support material into the cannula and into the patient,
spinning and advancing the cutting shaft past the distal end of the cannula within the patient, and
creating a cavity in tissue within the patient using the spinning cutting shaft.

22. The method of claim 21, wherein advancing the cutting shaft past the distal end of the cannula allows the cutting shaft to assume a second configuration.

23. The method of claim 21, wherein spinning and advancing the cutting shaft past the distal end of the cannula allows the cutting shaft to assume a second configuration.
